(19) United States
(12) Patent Application Publication
(10) Pub. No.: US 2007/0086986 A1
(43) Pub. Date: Apr. 19, 2007

(54) Preparation of three-dimensional mammalian ovarian follicular cell and ovarin follicle culture systems in a biocompatible matrix

(76) Inventors: Daniele Vigo, Milano (IT); Vincenzo Russo, Milano (IT); Massimo Faustini, Milano (IT); Simona Stacchezzini, Milano (IT); Ubaldo Conte, Pavia (IT); Maria Luisa Torre, Pavia (IT); Pier Attilio Accorsi, Bologna (IT); Giovanna Galeati, Bologna (IT); Marcella Spinaci, Bologna (IT)

Correspondence Address: DICKSTEIN SHAPIRO LLP, 1825 EYE STREET NW, Washington, DC, 20006-5403, US

(21) Appl. No.: 10/577,806

(22) PCT Filed: Nov. 2, 2004
(86) PCT No.: PCT/EP04/12384

§ 371 Date: Dec. 19, 2006

(30) Foreign Application Priority Data
Nov. 3, 2003 (IT) MI2003A002115

(51) Int. Cl. A61K 35/12; C12N 5/08; A61K 9/48

(52) U.S. Cl. 424/93.7; 424/451; 435/366

(57) ABSTRACT

A process for encapsulating and immobilising mammalian stem cells, ovarian follicular cells, gametes, ovarian follicles or mammalian embryos which are able to auto-organise into three-dimensional structures in vitro, and express biological functions in a manner similar to that which is observed in the organism in vivo is described. The capsules are constituted by: a nucleus containing stem cells, ovarian follicular cells, gametes ovarian follicles or mammalian embryos and/or a biocompatible and/or biodegradable polymer; a semi-permeable membrane constituted by a divalent or trivalent metal ion salt of alginic acid, optionally cross-linked on the inner and/or outer surface and/or on both surfaces, optionally vehicularising a second or more cellular species. The cells and follicles prepared and cultivated using this methodology are used for the in vitro and/or in vivo production of peptides, proteins, antibodies, hormones and hormone precursors, metabolites and catabolites typical of these cellular structures, plasma membranes, nuclear membranes, cytoplasmic organelles, somatic cell nuclei or gametes and embryos.

### FIELD OF THE INVENTION

The present invention relates to semi-permeable membrane capsules containing cells or follicles of various types for the preparation of organs, tissues or biological substances both in vitro and in vivo.

In recent years there has been great interest in the study of novel technologies suitable for the encapsulation within semi-permeable and biocompatible living cell membranes, with the aim of transplanting cells, tissues or tissue parts into living organisms without resorting to the use of immunosuppressant drugs (Uludag et al., 2000). Currently, the culture of isolated living cells is performed predominantly in liquid media or on mono-layers on suitable culture dishes whilst maintaining appropriate conditions of temperature and humidity. Both the above methods may only simulate, in a very limited manner, the complexity of an entire organism since the cells are deprived of their tissue specific extracellular matrix. During culture, in the absence of extracellular matrix, the cells frequently undergo alterations to their morphology and their biochemical and functional properties, above all due to the adhesion of the cells to an unsuitable substrate, an inadequate supply of nutrients and two dimensional growth conditions. (Sittinger et al., 1996). In their natural environment, cells are found in a complex three-dimensional system constituted by an intricate network of proteins and polysaccharides which plays a dynamic role in the regulation of cellular functionality (Li, 1998). Hence, in order to achieve the development of cells or tissues in vitro, the formation of an extracellular matrix, as close to that found physiologically, allowing the three-dimensional organisation of the cells, is indispensable. Such an arrangement, potentially similar to that found in living tissues is able to obviate the aggregation of the cells into dense clumps with the consequent loss of efficiency and functionality.

Many authors have used different types of polymer-based matrices (scaffolds), in order to allow the development of isolated cells in vitro. Such matrices have high porosity and are able to provide attachment sites suitable for the orientation and growth of a sufficient number of cells, so as to guarantee survival and functionality, similar to that found in vivo (Shapiro and Cohen, 1997). In order to achieve adequate growth of the cells, the structural uniformity of the polymeric scaffold, which must be constituted by biocompatible materials with appropriate mechanical characteristics (Kuo and Ma, 2001) is necessary.

A different approach for the attainment of three dimensional culture systems is the encapsulation of cells, by entrapping a population of living cells inside an artificial extracellular matrix bounded by semi-permeable membranes, thus physically isolating them from the external environment. The extracellular matrix within the capsule is essential so that the cells auto-organise themselves into structures functionally similar to tissues in vivo.

It was Chang who succeeded in obtaining “artificial cells”: systems constituted by polymeric materials, suitable for encapsulating proteins, enzymes or cells (Chang, 1964). One of the first applications has been the vehicularisation of pancreatic cells in alginate capsules for the treatment of diabetes (Lim and Sun, 1980). Cells or tissues were suspended in sodium alginate, and such suspension was extruded into a solution containing bivalent cations, such as calcium ions: the ions bring about the polymerisation of the polymer and the transformation of the suspension into a rigid matrix (bead). Through subsequent treatment with a solution of poly-L-lysine, a permanent semi-permeable membrane forms on the surface of the capsules, the porosity of which could be adjusted depending on the molecular weight and concentration of the poly-L-lysine, and depending on the concentration and type of alginate used (De Vos et al., 1993).

Recently, Mauchamp et al. (1998) have found that isolated porcine thyroid follicular cells organise themselves into pseudofollicles if they are allowed to adhere onto a type-I collagen matrix. Such structures are not obtained with cell cultures in monolayers.

Adequate permeability of the polymeric membrane (cut off) is indispensable for the survival and auto-organisation of encapsulated living cells. The ideal membrane should allow the entry of molecules essential for the survival of the cells and the elimination of secreted substances and the waste substances from cellular metabolism (Colton, 1996); further, it should result in a state of immuno-isolation, inhibiting the entry of effectors of the organism's immune response into the cellular environment.

Semi-permeable membranes, with precise molecular cut-offs, allow the diffusion of cellular secretions, catabolites and metabolites. The permeability and selectivity of the membranes thus represent a first critical aspect in the development of such types of systems. Adequate mechanical properties for the capsules, both in terms of resistance to breakage, and in terms of elasticity, size distribution and surface properties are indispensable.

Primordial ovarian follicles are structures characterised by a single layer of flat cells, similar to epithelial cells: such cells, during the maturation of the follicles, become cuboidal in shape and begin to divide, differentiating into outer theca cells, inner theca and the granulosa. During the entire in vivo maturation period giving rise to the Graaf follicle, the granulosa cells are able to produce predominantly oestrogens through aromatase enzyme system, which uses androgens and progesterone as substrates. Following the ovulation process, the granulosa cells differentiate morphologically and functionally moving towards progesterone biosynthesis.

Recently, a novel living cell encapsulation technology, particularly for porcine spermatozoa (EP0922451), has been developed. Divalent ions are added to the seminal material and such suspension is extruded into an aqueous solution of sodium alginate. Upon contact with the alginate solution the divalent ions diffuse towards the outer surface thus inducing the gelification of the alginate around the cellular suspension. Such capsules may have their outer surfaces cross-linked using polyamines, such as for example protamine, thus altering the mechanical properties and the permeability of the membrane.

The advantage of this technology with respect to other encapsulation and micro-encapsulation technologies is that the process steps are reduced and the cells thus contained do not undergo any chemical or physical stresses which would compromise their functionality and structure.

### DETAILED DESCRIPTION OF THE INVENTION

To date, no attempts have been reported in the literature of the encapsulation of mammalian ovarian follicular cells or ovarian follicles. The use and culture of ovarian follicles and granulosa cells of bovines, equines, caprines, porcines, canines, felines, lagomorphs, mouse and rat and laboratory species in general, as well as humans, but preferably porcines and bovines, is particularly interesting in that such cells, when suitably cultivated, produce hormones or proteins and/or biologically active substances analogously to those which said cells are able to produce in vivo. These physiologically produced substances contribute towards the maturation of the oocyte.

The present invention relates to an encapsulation technology for ovarian follicular cells, mature and immature gametes, embryos and ovarian follicles at various stages of mammalian development in a biocompatible matrix, enclosed within a membrane of a divalent or trivalent metal salt of alginic acid, optionally cross-linked on the inner and/or outer surface and/or on both surfaces. Besides the aforementioned cellular species, stem cells of various origins may be vehicularised within the capsules; indeed, the latter show morphological and functional characteristics similar to the granulosa cells which constitute the primordial follicles. Further, genetically modified male and female somatic cells may be vehicularised within the capsules, for example pancreatic and thyroidal cells. Cells, tissue or organ parts, tissues or organs, gametes or embryos may be preserved whilst awaiting encapsulation at laboratory temperature, or by refrigeration, freezing, cryopreservation or lyophilisation.

The cells vehicularised within the capsules auto-arrange themselves in vitro into three-dimensional follicular, parenchymatose or alveolar structures, which permit the in vitro growth of tissues and multicellular structures functionally similar to the organs within the whole organism.

Said cellular structures express biological functions which may not be currently reproduced in vitro with other cell culture technologies. The capsule structure allows the attainment of a microenvironment similar to that found physiologically, characterised by the presence of an extracellular matrix and a semi-permeable membrane which acts as a basal membrane.

The cell cultures obtained using this methodology are useful for the production of peptides, proteins, hormones, for the biological assay of drugs, hormones and hormone precursors, for the evaluation of the efficacy of drugs and the toxicity and teratogenicity of chemical and pharmacological substances, for improving the in vitro yields of oocell, follicle and embryo cultures and co-cultures in experimental practices and reproductive biotechnology applications. Further, such cell cultures may be implanted into individuals as hormonal-type replacement therapies, indeed the polymeric film (i.e. the membrane coating the capsule) which surrounds the artificial tissue, vehicularised within the capsule, constitutes an immuno-protective barrier which allows the obviation of the use of immunosuppressive drugs.

Particularly, the encapsulated mammalian ovarian follicular cells and ovarian follicles are capable of producing progesterone (P4) and 17β-oestradiol (E2) analogously to that which occurs in vivo.

The capsules are essentially constituted by:
a nucleus containing mammalian stem cells, ovarian follicular cells, gametes and embryos or ovarian follicles and/or a biocompatible and/or biodegradable polymer;
by a semi-permeable membrane constituted by a divalent or trivalent metal salt of a biocompatible and biodegradable polymer such as for example alginic acid, optionally cross-linked on its inner and/or outer surface and/or on both surfaces, optionally vehicularising a second cellular type.

Within said nucleus the cells are suspended in a gelatinous medium.

The organs or tissues are removed from various mammalian species, such as bovines, equines, caprines, lagomorphs, porcines, canines, felines, rodents and possibly humans, but preferably from porcines and bovines. Such removal may be carried out at the time of slaughter, during the removal of biopsy material or whilst performing surgical operations, but for livestock preferably at the time of normal slaughter. The tissues or organs of interest are removed, preferably the female gonads.

In the case where the organs of interest are the ovaries, these are appropriately removed and washed in a physiological solution, as known to experts of the art.

The somatic cells within the follicle and the gametes are isolated from the tissues by aspiration, centrifugation of the follicular liquids, or digestion of the intracellular matrix as known to those skilled in the art. Following centrifugation, the cellular sediment is washed by repeated passages in culture medium and recovered by removal of the supernatant. The cellular concentration of the sediment is determined by direct counting using a Makler chamber, or Bürker chamber, or by cytofluorimetry, or by using semi-automated and automated cell counters.

The isolated cells may be suspended in culture or maintenance media until their encapsulation, preserving them in an environment at a temperature between room temperature and −200° C. and humidity between 40% and 100%, as known to those skilled in the art.

As culture or maintenance media, the followings may be used: physiological solution (isotonic saline), glucosate solution, Basal Medium Eagle (BME) and derivatives thereof, Hanks salts solution and derivatives thereof, tissue culture medium 199 (TCM 199) and derivatives thereof, phosphate buffered saline (PBS) and derivatives thereof, Krebs salts solution and derivatives thereof, Dulbecco modified Eagle's medium (DMEM) and derivatives thereof, tris-buffered medium (TBM) and derivatives thereof, Tyrode's salts solution and derivatives thereof, Modified sperm washing medium, modified human tubal fluid, Modified ham's F-10 medium, Upgraded B2 INRA medium, B2 INRA Menezo Medium, Upgraded B9 medium and various other culture media specifically used by those skilled in the art, but preferably TCM 199 and derivatives thereof as well known to any specialist skilled in the art.

According to the present invention, the cells, suspended in culture medium or follicular liquid, may be optionally diluted into a culture medium containing a hydrophilic polymer which constitutes the artificial extracellular matrix. The cellular sediment dilution-polymeric solution ratio may be between 1:0.05 and 1:200, and preferably between 1:0.1 to 1:100.

The polymeric material constituting the artificial extracellular matrix of the nucleus of the capsules, forming the subject of the present invention is preferably selected from the group constituted by: glucans, scleroglucans, mannans, galactomannans, gellans, carrageenans, pectins, polyanhydrides, polyaminoacids, polyamines, xanthans, celluloses and derivatives thereof, carboxymethylcellulose, ethylcellulose, methylcellulose, hydroxypropylcellulose hydroxypropylmethylcellulose, polyvinylalcohols, carboxyvinylpolymers, starches, collagens, chitins, chitosans, alginic acid, hyaluronic acid. Such polymers, in aqueous solution, at an appropriate pH value, which depends on the nature of the polymer, as known to those skilled in the art, are generally used in concentrations between 0.01% and 90% of total capsule weight, but preferably between 0.5% and 50%. Preferably, xanthan gum at various viscosities, generally between 800 cP and 1200 cP, is used as artificial extracellular matrix.

The capsule membrane, forming the subject of the present invention, is generally constituted by alginates of divalent metals such as calcium, barium, strontium, zinc and trivalent metals such as aluminium, iron and chromium.

In the preparation of the capsules, forming the subject of the present invention, to the cell suspension is added a divalent or trivalent ion, such ion is added, preferably as a chloride or sulphate in solution, so as to obtain cation concentrations of between 1 and 500 mmol/l and preferably between 5 and 200 mmol/l. The extruded cellular suspension and the alginate solution volume ratio may be between 1:1 and 1:250, and preferably between 1:15 and 1:50.

The cellular suspension is subsequently extruded through extruders, orifices, nozzles or needles, having dimensions between 50 μm and 5000 μm, preferably through needles having internal diameters between 300 μm and 2000 μm into a solution of sodium alginate in medium, whilst kept stirring, at speeds between 10 and 200 rpm, but preferably between 20 and 100 rpm. The alginates used in the preparation of the capsules forming the subject of the present invention have, in a 2% solution in water, a viscosity between 200 cP and 20000 cP at 25° C. The alginate concentration in the solutions is between 0.01% and 5% w/v, but preferably between 0.1% and 1%.

The presence of divalent and trivalent ions in the extruded cellular suspension induces the gelification of the alginate at the droplet interface and the formation of a gelatinous membrane with the consequent attainment of the capsule.

Such operations are performed at temperatures between 5° C. and 40° C., and preferably at 20-30° C.; extrusion occurs using automated or semi-automated microencapsulators, peristaltic or piston pumps or alternatives, or using a manually operated syringe, or appropriate system, at such a speed as to produce between 10 to 250 drops/minute, and preferably 60 drops/minute.

Said capsules may be subjected to cross-linking, by interfacial polymerisation of the alginate using polyamine based cross-linking agents, such as for example: protamine sulphate or phosphate, preferably in solution at concentrations between 0.01% and 5% w/v, or poly-L-lysine bromohydrate having a molecular weight between 1000 Da and 800000 Da in solution at a concentration preferably between 0.01% and 5% w/v, or polyvinylamine at a concentration of from 0.01% to 5% w/v, or chitosans of molecular weight between 15000 Da and 1,000,000 Da in concentrations between 0.01% and 5% w/v.

The cross-linking reaction is carried out at a temperature between 5° C. and 40° C., and preferably at 25° C. for times between 1 minute and 120 minutes, preferably between 3 and 30 minutes. This procedure causes the conversion of the gelatinous membrane into a semi-permeable rigid membrane of cross-linked alginate. Said capsules have a cross-linked membrane and are recovered by filtration, washed and suspended in an appropriate maintenance medium as known to those skilled in the art.

Spheroidal shaped capsules are obtained having S dimensions between 0.5 and 30 mm, but preferably between 2 mm and 10 mm, with membrane thicknesses between 300 μm and 5000 μm. The weight of the capsule produced is between 5 mg and 200 mg, bur preferably between 20 mg and 100 mg.

Said capsules, suspended in medium, are preserved at temperatures between −200° C. and 40° C. but preferably between 4° C. and 40° C., and still more preferably at 38.5° C., optionally in a controlled atmosphere, as known to those skilled in the art.

Using disposable, preset and pre-packaged instrumentation, for single and/or multiple preparations, capsules may be prepared by starting from previously prepared, pre-measured and pre-packaged raw materials.

Hence, a further subject of the present invention is a kit for the preparation of capsules, according to the invention, comprising previously prepared, pre-measured and pre-packaged raw material, as well as the relevant disposable, sterile, non sterile or sterilisable materials. The preparation of the capsules may be performed by vehicularising into said capsules: cells, tissues, tissue parts, organs, organ parts, cell cores, gametes and embryos, freshly removed and/or appropriately preserved according to the techniques known to those skilled in the art.

Capsules containing cell cores, tissues, organs or parts thereof, gametes or embryos may be co-incubated, in an appropriate culture medium, with other cell cores, tissues, organs or parts thereof, gametes and embryos, thus encouraging the development of cell cores, tissues, organs or parts thereof, gametes and embryos under conditions simulating the physiological environment.

The biosynthesis of specific products and/or specific biologically active substances is favoured under such conditions. The incubation and/or the co-incubation allow the encapsulated biological structures to produce hormones, metabolites, catabolites and other biologically active substances.

The metabolites, catabolites and the biologically active substances produced or secreted and synthesised by the encapsulated structures may be recovered from the culture medium and/or from within the capsules by aspiration, or removed using techniques known to those skilled in the art.

Said metabolic, catabolic and secretory products may be extracted, purified and appropriately characterised as known to those skilled in the art, without irreversibly damaging the three-dimensional capsular culture system.

Said products may be used directly or following purification or concentration, in order to modulate growth, development, maturation and functionality, of other cells, tissues, organs, gametes and embryos, in other in vitro culture systems and/or in ex vivo, and/or in vivo systems.

Analogously, according to the known art, cell cores, autologous or heterologous tissues or organs or parts thereof, as well as gametes and embryos at various stages of development, may be injected, microinjected, inserted within the capsules, without irreversibly damaging the three-dimensional capsular culture system.

Cell cores, tissues or organs or parts thereof, gametes and embryos may be aspirated or removed from said capsules at pre-arranged times using means and techniques known to those skilled in the art, without irreversibly damaging the three-dimensional capsular culture system. The following examples are reported for the purpose of non-limiting illustration of the capsule preparation process forming the subject of the present invention.

### EXAMPLE 1

### Encapsulation and Three-Dimensional Culture of Bovine Granulosa Cells

1a) Preparation of the Cells

The ovaries at various stages of the oestrous cycle are removed from cows, starting from 16-18 months of age, during normal slaughter, washed with physiological solution at 30° C., as known to those skilled in the art. Follicles having a diameter of 2-6 mm are identified in the ovaries, from which the follicular liquids, containing the granulosa cells, are aspirated using syringes. The cellular suspensions thus obtained are centrifuged and washed twice with 10 ml of TCM199 medium+10% foetal calf serum+1% penicillin/streptomycin. Following centrifugation, a cellular sediment is obtained, the cellular concentration of which is determined by direct counting using a Makler chamber.

1b) Encapsulation

The cellular sediment is diluted in a solution of xanthan gum (Satiaxane®, SKW Biosystems, France) at 0.5% in TCM199 culture medium containing Earle salts, L-glutamine and sodium bicarbonate (Sigma-Aldrich,); the cellular sediment to xanthan gum solution volume ratio is 1:3. A cellular suspension is obtained, to which is then added a saturated barium chloride solution up to a final concentration of 20 mmol/l of barium ions. The resulting suspension is extruded through needles (26G×½″, 0.45×13 mm) into a medium viscosity (3500 cP,) sodium alginate solution at 0.5% w/v in culture medium, kept stirring using a magnetic stirrer (30 rpm). The cellular suspension to sodium alginate solution volume ratio is 1:25. The extrusion takes place dropwise through the syringe, at a temperature of 25° C. The barium ions react with the sodium alginate forming a barium alginate membrane at the interface of the individual drops of extrudate within 30′. Capsules are obtained, which are collected by filtration, washed twice with culture medium and suspended in an aliquot of the same. Said capsules are subsequently cross-linked on their external surfaces using a 1% solution of protamine sulphate (Sigma-Aldrich, Milan, Italy) in TCM199 culture medium containing Earle salts, L-glutamine and sodium bicarbonate (Sigma-Aldrich,) for 30 minutes at a temperature of 25° C.

The population of granulosa cells is found inside the cross-linked capsule, in an artificial extracellular matrix.

Spheroidal shaped capsules are obtained having dimensions between 2 mm and 10 mm and weights between 20 mg and 100 mg. The capsules thus produced may be preserved, under normal laboratory conditions, in specific controlled environment incubators, by lyophilisation, refrigeration, freezing or cryopreservation.

1c) Three-Dimensional Cell Culture

A capsule is placed in a sterile cell culture plate well suspended in 600 μl of culture medium (TCM199 containing foetal calf serum (10%), penicillin/streptomycin (1%) and 3-17androstenedione (100 ng/μl).

The plates containing the capsules are maintained in an incubator for 6 days at 38.5° C., 5% CO₂ and 90% humidity.

Every 48 hours, from each well, samples of the medium containing the cellular metabolic products are taken; the samples are promptly frozen in Eppendorf tubes, at a temperature of less than −20° C.

In the wells containing the capsules, the culture medium is substituted with an equal volume of fresh medium, with the continuation of the culture on the same sample.

Hence, the steroidogenic activity, in terms of the production of progesterone (P4) and 17β-oestradiol (E2), has been evaluated in each sample of medium removed from the wells by radioimmuno assay (RIA).

The results are expressed as the ratio between P4 and E2, known to those skilled in the art as the luteinisation index.

TABLE 1
Luteinisation index (P4/E2), standard deviation and sample number
of the bovine granulosa cells cultivated in the capsules.

| Days | Mean | Std. Dev. | N |
|---|---|---|---|
| 2 | 5.1 | 32.0 | 39 |
| 4 | 567.9 | 2245.3 | 35 |
| 6 | 9452.5 | 18254.4 | 23 |

From the results reported in table 1 it is deduced that cellular vitality is maintained in the encapsulated cells, with the production of both hormones throughout the entire culture period: The encapsulated cells produce low quantities of progesterone as observed in vivo in the follicle prior to ovulation.

That indicates reduced luteinisation of the encapsulated cells, which can only occur in follicular structures very similar to those found in vivo.

The information derived from analysis of the results underline that the bovine granulosa cells, encapsulated according to the process of the present invention, have steroidal activity analogous to that in vivo and obtainable only with a three-dimensional type cell culture process.

### Reference Example 1

In parallel, cell culture has been carried out in monolayers, in this case also evaluating the steroidogenic activity in terms of the production of progesterone (P4) and 17β-oestradiol (E2); the concentrations of such hormones in the samples of medium withdrawn from the wells have been evaluated using radioimmuno assay (RIA).

Non-encapsulated cells are seeded and cultivated in monolayers in welled plates, each containing 600 μl of the culture medium also used for the culture of the encapsulated cells.

Analogously to that described for the encapsulated cells, the plates containing the cells in monolayers are maintained in an incubator for 6 days at 38.5° C., 5% CO₂ and 90% humidity.

Every 48 hours, from each well, samples of the medium containing the cellular metabolic products are taken; the samples are frozen in Eppendorf tubes, at a temperature of less than −20° C. From the wells containing the cells cultivated in monolayers, the culture medium is completely removed and substituted with fresh medium, with the continuation of the culture on the same sample. The results obtained are reported in table 2.

TABLE 2
Luteinisation index (P4/E2), standard deviation and sample number
of the bovine granulosa cells cultivated in monolayers.

| Days | Mean | Std. Dev. | N |
|---|---|---|---|
| 2 | 7.4 | 38.5 | 27 |
| 4 | 1700.0 | 3870.9 | 23 |
| 6 | 70201.0 | 131436.5 | 11 |

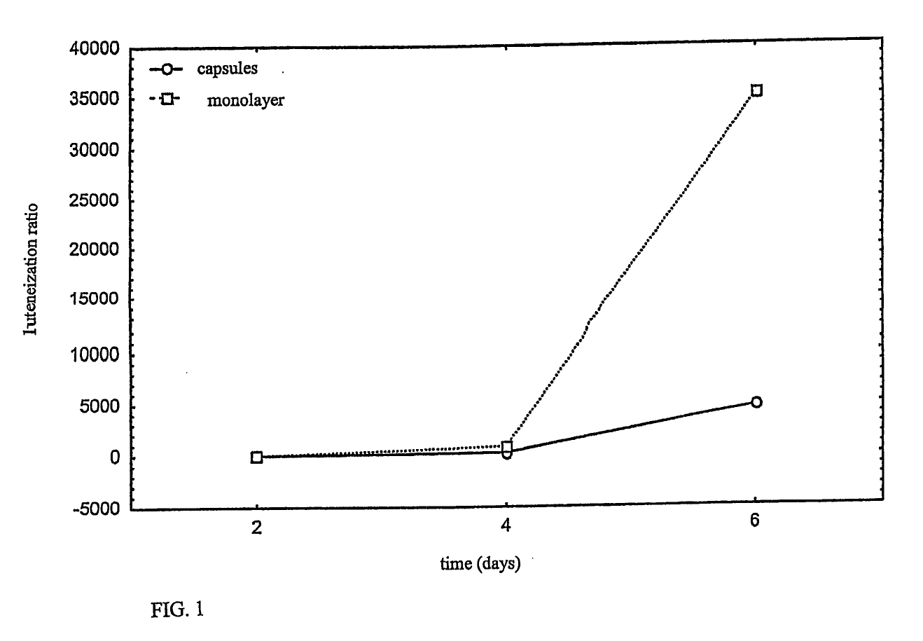

In FIG. 1 are reported the luteinisation indices of the bovine granulosa cells cultivated in monolayers and in the capsules as a function of culture time.

From the results reported in FIG. 1 it may be deduced that cellular vitality is maintained with both culture techniques, with the production of both hormones for the entire culture period.

Regarding the progesterone synthesised by the cells cultivated in monolayers, a significant increase in its concentration is observed on the 6^{th} day of culture: this increase is an indication of marked cellular luteinisation.

Such increase is less evident for the encapsulated cells which produce low quantities of progesterone as observed in vivo in the follicle prior to ovulation.

That indicates reduced luteinisation of the encapsulated cells and can only occur in follicular-like structures very similar to those found in vivo.

### EXAMPLE 2

### Encapsulation and Three-Dimensional Culture of Porcine Granulosa Cells

2a) Preparation of the Cells

The ovaries at various stages of the oestrous cycle are removed from subjects, starting from 6-11 months of age, during normal slaughter, washed with physiological solution at 30° C., as known to those skilled in the art. Follicles having a diameter of 2-6 mm are identified in the ovaries, from which the follicular liquids, containing the granulosa cells, are aspirated using syringes. The cellular suspensions thus obtained are centrifuged and washed twice with 10 ml of TCM199 medium+10% foetal calf serum+1% penicillin/streptomycin. Following centrifugation, a cellular sediment is obtained, the cellular concentration of which is determined by direct counting using a Makler chamber.

2b) Encapsulation

The cellular sediment is diluted in a solution of xanthan gum (Satiaxane®, SKW Biosystems, France) at 0.5% in TCM199 culture medium containing Earle salts, L-glutamine and sodium bicarbonate (Sigma-Aldrich,); the cellular sediment to xanthan gum solution volume ratio is 1:3. A cellular suspension is obtained, to which is then added a saturated barium chloride solution up to a final concentration of 20 mmol/l of barium ions. The resulting suspension is extruded through needles (26G×½″, 0.45×13 mm) into a medium viscosity (3500 cP,) sodium alginate solution at 0.5% w/v in culture medium, kept stirring using a magnetic stirrer (30 rpm). The cellular suspension to sodium alginate solution volume ratio is 1:25. The extrusion takes place dropwise through the syringe, at a temperature of 25° C. The barium ions react with the sodium alginate forming a barium alginate membrane at the interface of the individual drops of extrudate within 30′. Capsules are obtained, which are collected by filtration, washed twice with culture medium and suspended in an aliquot of the same. Said capsules are subsequently cross-linked on their external surfaces using a 1% solution of protamine sulphate (Sigma-Aldrich, Milan, Italy) in TCM199 culture medium containing Earle salts, L-glutamine and sodium bicarbonate (Sigma-Aldrich,) for 30 minutes at a temperature of 25° C.

The population of granulosa cells is found inside the cross-linked capsule, in an artificial extracellular matrix.

Spheroidal shaped capsules are obtained having dimensions between 2 mm and 10 mm and weights between 20 mg and 100 mg. The capsules thus produced may be preserved, under normal laboratory conditions, in specific controlled environment incubators, by lyophilisation, refrigeration, freezing or cryopreservation.

2c) Three-Dimensional Cell Culture

A capsule is placed in a sterile cell culture plate well suspended in 600 μl of culture medium (TCM199 containing foetal calf serum (10%), penicillin/streptomycin (1%) and 3-17androstenedione (100 ng/μl)).

The plates containing the capsules are maintained in an incubator for 6 days at 38.5° C., 5% CO₂ and 90% humidity.

Every 48 hours, from each well, samples of the medium containing the cellular metabolic products are taken; the samples are promptly frozen in Eppendorf tubes, at a temperature of less than −20° C.

In the wells containing the capsules, the culture medium is substituted with an equal volume of fresh medium, with the continuation of the culture on the same sample.

Hence, the steroidogenic activity, in terms of the production of progesterone (P4) and 17β-oestradiol (E2), has been evaluated in each sample of medium removed from the wells by radioimmuno assay (RIA).

The results are expressed as the ratio between P4 and E2, known to those skilled in the art as the luteinisation index.

TABLE 3
Luteinisation index (P4/E2), standard deviation and sample number
of the porcine granulosa cells cultivated in the capsules.

| Days | Mean | Std. Dev. | N |
|---|---|---|---|
| 2 | 14.8 | 55.3 | 38 |
| 4 | 124.0 | 338.2 | 30 |
| 6 | 43.7 | 83.8 | 20 |

From the results reported in table 3 it may be deduced that cellular vitality is maintained in the encapsulated cells, with the production of both hormones throughout the entire culture period: The encapsulated cells produce low quantities of progesterone as observed in vivo in the follicle prior to ovulation.

That indicates reduced luteinisation of the encapsulated cells, which can only occur in follicular structures very similar to those found in vivo.

### Reference Example 2

In parallel, cell culture has been carried out in monolayers, in this case also evaluating the steroidogenic activity in terms of the production of progesterone (P4) and 17β-oestradiol (E2); the concentrations of such hormones in the samples of medium withdrawn from the wells have been evaluated using radioimmuno assay (RIA).

Non-encapsulated cells are seeded and cultivated in monolayers in welled plates, each containing 600 μl of the culture medium also used for the culture of the encapsulated cells. Analogously to that described for the encapsulated cells, the plates containing the cells in monolayers are maintained in an incubator for 6 days at 38.5° C., 5% CO₂ and 90% humidity.

Every 48 hours, from each well, samples of the medium containing the cellular metabolic products are taken; the samples are frozen in Eppendorf tubes, at a temperature of less than −20° C. From the wells containing the cells cultivated in monolayers, the culture medium is completely removed and substituted with fresh medium, with the continuation of the culture on the same sample. The results obtained are reported in table 4.

TABLE 4
Luteinisation index (P4/E2), standard deviation and sample number
of the porcine granulosa cells cultivated in monolayers.

| Days | Mean | Std. Dev. | N |
|---|---|---|---|
| 2 | 2.5 | 2.3 | 36 |
| 4 | 22.1 | 23.4 | 25 |
| 6 | 2160.9 | 4997.9 | 24 |

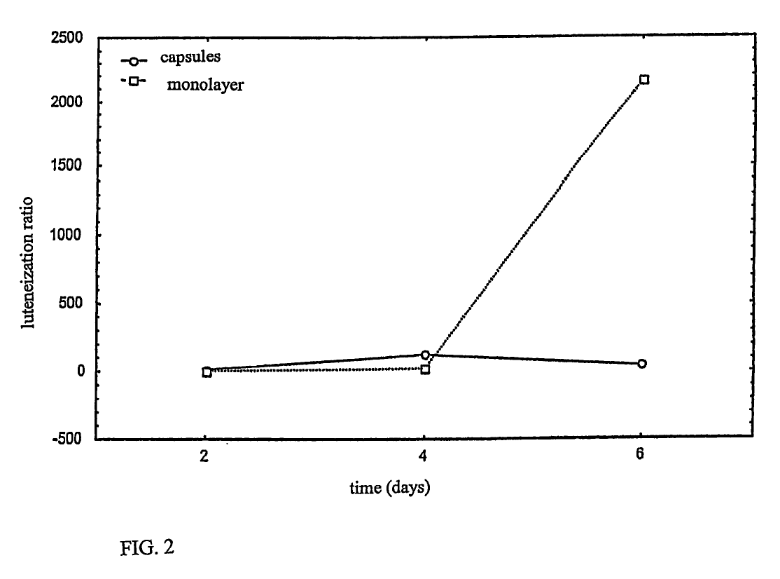

In FIG. 2 are reported the luteinisation indices of the porcine granulosa cells cultivated in monolayers and in the capsules as a function of culture time.

The information derived from analysis of the results underline that the porcine granulosa cells, encapsulated according to the process of the present invention, have steroidal activity analogous to that in vivo and obtainable only with a thee-dimensional type cell culture process.

1. Capsules comprising an outer membrane of a divalent or trivalent metal ion salt of alginic acid, and an internal nucleus comprising a suspension of stem cells, genetically modified male and female somatic cells, ovarian follicular cells, gametes, ovarian follicles, mammalian embryos and/or a biocompatible and/or biodegradable polymer constituting an artificial extracellular matrix.

2. The capsules according to claim 1, wherein said cells are suspended in a gelatinous medium.

3. The capsules according to claim 1 characterised in that said mammalian stem cells, genetically modified male and female somatic cells, ovarian follicular cells, ovarian follicles, gametes, and mammalian embryos are auto-organised in vitro into three-dimensional parenchymatose, follicular or alveolar type structures, which allow the in vitro growth of tissues and multicellular structures functionally similar to organs present in the whole mammalian organism.

4. The capsules according to claim 1, characterised in that said stem cells, genetically modified male and female somatic cells, ovarian follicular cells, gametes, ovarian follicles and mammalian embryos are functionally similar to the organs found in the whole organism and able to synthesise and secrete hormones such as progesterone (P4) and 17a-oestradiol (E2) and other biologically active substances in quantities similar to that which said structures produce in vivo.

5. The capsules according to claim 1 wherein said alginate membrane is gelatinous, bioerodible and is cross-linked on the inner and/or outer surfaces and/or on both surfaces.

6. The capsules according to claim 1 characterised in that the alginate membrane may be cross-linked using cross-linking agents selected from: protamine sulphate or phosphate, poly-L-lysine bromohydrate, polyvinylamine, or chitosans.

7. The capsules according to claim 1 wherein the outer membrane is constituted by divalent metal alginates selected from: calcium, barium, strontium, zinc or trivalent metals selected from: aluminium, iron, chromium.

8. The capsules according to claim 1 characterised in that the alginate membrane contains a second or more cellular species.

9. The capsules according to claim 8 characterised in that the alginate membrane contains ovarian follicular cells, one or more gametes, one or more ovarian follicles and one or more mammalian embryos.

10. The capsules according to claim 8 characterised in that the alginate membrane contains one or more female gametes also in various stages of development.

11. The capsules according to claim 1 characterised in that said biocompatible and/or biodegradable polymer is a hydrophilic polymer selected from the group constituted by: glucans, scleroglucans, mannans, galactomannans, gellans, carrageenans, pectins, polyanhydrides, polyaminoacids, polyamines, xanthans, celluloses and derivatives thereof: carboxymethylcelluloses, ethylcelluloses, methylcelluloses, hydroxypropylcelluloses hydroxypropylmethylcelluloses, polyvinylalcohols, carboxyvinylpolymers, starches, alpha, beta, gamma cyclodextrins and dextrin derivatives in general, collagens, chitins, chitosans, alginic acid, hyaluronic acid.

12. The capsules according to claim 11, characterised in that said polymers, in aqueous solution, are present in concentrations between 0.01% and 90% of the total capsule weight.

13. The capsules according to claim 12, characterised in that said polymers, in aqueous solution, are present in concentrations between 0.5% and 50% of the total capsule weight.

14. The capsules according to claim 12, characterised in that the hydrophilic polymeric material, which constitutes the artificial extracellular matrix of said capsule nucleus, is xanthan gum having viscosity between 800 and 1200 cP.

15. The capsules according to claim 1 characterised in that said capsules have diameters between 0.5 mm and 30 mm, with membrane thicknesses between 300 pm and 5000 urn.

16. The capsules according to claim 15 characterised in that said capsules preferably have diameters between 2 mm and 10 mm.

17. The capsules according to claim 1 characterised in that said capsules weigh between 5 mg and 200 mg.

18. The capsules according to claim 17 characterised in that said capsules preferably weigh between 20 mg and 100 mg.

19. A kit for the preparation of capsules according to claim 1 comprising disposable, sterile, non-sterile or sterilisable, preset and pre-packaged instrumentation, for single and or multiple preparations.

20. The kit according to claim 19 comprising salts of a divalent or trivalent ion, an alkaline metal alginate, in separate pre-measured packages.

21. The kit according to claim 20, further comprising a biocompatible and/or biodegradable hydrophilic polymer preferably selected from: glucans, scleroglucans, mannans, galactomannans, gellans, carrageenans, pectins, polyanhydrides, polyaminoacids, polyamines, xanthans, celluloses and derivatives thereof: carboxymethylcelluloses, ethylcelluloses, methylcelluloses, hydroxypropylcelluloses hydroxypropylmethylcelluloses, polyvinylalcohols, carboxyvinylpolymers, starches, alpha, beta, gamma cyclodextrins and dextrin derivatives in general, collagens, chitins, chitosans, alginic acid, hyaluronic acid.

22. The kit according to claim 20, further comprising a cross-linking agent preferably selected from protamine sulphate or phosphate, poly-L-lysine bromohydrate, polyvinylamine, or chitosans.

23. The kit according to claim 20, further comprising a culture medium preferably selected from: physiological solution (isotonic saline), glucosate solution, Basal Medium Eagle (BME) and derivatives thereof, Hanks salts solution and derivatives thereof, tissue culture medium 199 (TCM 199) and derivatives thereof, phosphate buffered saline (PBS) and derivatives thereof, Krebs salts solution and derivatives thereof, Dulbecco modified Eagle's medium (DMEM) and derivatives thereof, tris-buffered medium (TBM) and derivatives thereof, Tyrode's salts solution and derivatives thereof, Modified sperm washing medium, modified human tubal fluid, Modified ham's F-10 medium, Upgraded B2 INRA medium, B2 INRA Menezo Medium, Upgraded B9 medium.

24. The kit according to claim 20, further comprising extrusion devices such as sterile, non-sterile or sterilisable nozzles, needles or syringes.

25. The kit according to claim 20, wherein said divalent or trivalent ion salts are selected from: salts of calcium, barium, strontium, zinc, aluminium, iron or chromium.

26. The kit according to claim 20, wherein said alginate is sodium alginate.

27. A process for the preparation of capsules according to claim 1 which comprises the following steps: a) suspension of the cells in a culture medium or in an appropriate biological liquid, optionally containing a biocompatible and/or biodegradable hydrophilic polymer; b) the addition to the suspension thus obtained of a divalent or trivalent ion salt until the attainment of ion concentrations between 1 and 500 mmol/l; c) extrusion of the cellular suspension through extruders, orifices, nozzles or needles having dimensions between 50 pm and 5000 um into an alkaline metal alginate solution in culture medium, having a concentration between 0.01% and 5% w/v, kept stirring at a speed between 10 and 200 rpm; d) optionally, cross-linking of the capsules thus formed, through interfacial polymerisation of the alginate using the cross-linking agents according to claim 5, at a temperature between 5 C and 40 C for a time between 1 minute and 120 minutes.

28. The process according to claim 27 further comprising the recovery stage of the capsules by filtration, the washing of the same and their suspension in culture medium.

29. The process according to claim 27 further comprising the preservation stage of the capsules under laboratory culture conditions, or by lyophilisation, refrigeration, freezing or cryopreservation.

30. The process according to claim 27 further comprising the vehicularisation stage within said capsules of cells, tissues, tissue parts, organs, organ parts, cell cores, gametes and embryos either freshly removed or appropriately preserved.

31. The process according to claim 30 wherein said vehicularisation stage comprises the stage of injecting or microinjecting into said capsules, cells, tissues, tissue parts, organs, organ parts, cell cores, gametes and embryos at various stages of development, either freshly removed or appropriately preserved.

32. The process according to claim 27 further comprising the incubation stage, in an appropriate culture medium, of said capsules with cell cores, tissues, organs or parts thereof, gametes and embryos.

33. The process according to claim 27 further comprising the stage of aspiration or removal, with any means or any technique and during any developmental stage of the cell cores, tissues or organs or parts thereof, gametes, embryos or substances produced by them.

34. The process according to claim 27 further comprising the stage of extraction, purification, characterisation and sequencing of the substances produced such as hormones, metabolites, catabolites and other biologically active substances.

35. The process according to claim 27 wherein, in step (a), said divalent or trivalent ion is calcium, barium, strontium, zinc, aluminium, iron or chromium chloride or sulphate at a concentration between 5 and 200 mmol/l.

36. The process according to claim 27 wherein, in step (a), the culture medium used is selected from: physiological solution (isotonic saline), glucosate solution, Basal Medium Eagle (BME) and derivatives thereof, Hanks salts solution and derivatives thereof, tissue culture medium 199 (TCM 199) and derivatives thereof, phosphate buffered saline (PBS) and derivatives thereof, Krebs salts solution and derivatives thereof, Dulbecco modified Eagle's medium (DMEM) and derivatives thereof, tris-buffered medium (TBM) and derivatives thereof, Tyrode's salts solution and derivatives thereof, Modified sperm washing medium, modified human tubal fluid, Modified ham's F-10 medium, Upgraded B2 INRA medium, B2 INRA Menezo Medium, Upgraded B9 medium, optionally containing a biocompatible and/or biodegradable polymer which constitutes the artificial extracellular matrix.

37. The process according to claim 27 wherein, in step (a), the medium used is TCM 199 and derivatives thereof, containing a hydrophilic polymer constituting the artificial extracellular matrix.

38. The process according to claim 27 wherein, in step (a), the cellular sediment dilution/polymeric solution volume ratio is between 1:0.05 and 1:200.

39. The process according to claim 38 wherein the cellular sediment dilution/polymeric solution volume ratio is between 1:0.1 to 1:100.

40. The process according to claim 27 wherein, in step (c), the extruded cellular suspension and the alginate solution volume ratio is between 1:1 and 1:250.

41. The process according to claim 27 wherein, in step (c), extrusion occurs through the use of automated, semi-automated microencapsulators, peristaltic or piston pumps or alternatives, or by the use of manually operated syringes at such a speed as to produce from 10 to 250 drops/minute.

42. The process according to claim 27 wherein, in step (c), the extrusion, with automated, semi-automated microencapsulators, peristaltic or piston pumps or alternatives, or by the use of manually operated syringes, occurs at such a speed as to produce 60 drops/minute.

43. The process according to claim 27 wherein, in step (c), the extrusion of the cellular suspension occurs through the use of needles with internal diameters between 300 pm and 2000 um.

44. The process according to claim 27 wherein, in step (c), the alginate solution is kept stirring at a speed between 20 and 100 rpm and has a concentration between 0.1% and 1% w/v.

45. The process according to claim 27 wherein, in step (c), the extruded cellular suspension and the alginate solution volume ratio is between 1:15 and 1:50.

46. The process according to claim 27 wherein said alginates, in a 2% solution in water and at a temperature of 25 C, have a viscosity between 200 cP and 20000 cP.

47. The process according to claim 27 wherein steps (a), (b) and (c) are performed at a temperature between 5 C and 40 C.

48. The process according to claim 27 wherein steps (a), (b) and (c) are performed at a temperature between 20 C and 30 C.

49. The process according to claim 27 wherein step (d) is performed at a temperature between 5 C and 40 C, for times between 1 minute and 120 minutes.

50. The process according to claim 27 wherein step (d) is performed at a temperature between 20 C and 30 C, for times between 3 minutes and 30 minutes.
